SEQUENCE LISTING (5 sequences)

SEQ ID NO: 1 - DNA, 910 bp, Populas trichocarpa nisqually1
catgggttgtctcttctctgggaactggttgaagtcaattataagaacgagaaaggcgaagaaagatgcatcaaagaaagtaaaggtacactcggctaccgaaaaggcaaacgggtcaaaggagagctcacccgcccatggagaatcaagtaatcttgccaatggtgatttggagagcaatattcatgttgctcctggattgtctgctgaatatatagctgctgtcaggattcaagatgcttttcgggcatacaaggcaagaaaagcaatgcaccgtcttaagggcgccgtgaggttcaatgtgctgattcatggtcatgacactcaaaagcaagcttcaagcacattaagccatattcattcatggagcaacatacaggcacagattagagcccgccggcaccatatggtcaccgaaggccgtatcaagcaaaagaaattagaaaaccagttgaaactcgaggccagacttcaagagattgaggtggagtggtgcggtggctccgataccatggaggaaatcctttccaggatccagcaaagagaagaagcagcagtaaagcgtgaacgagccatggcatatgccttctctcatcagtggagagccaaccctacccaatatctaggccaggcctattacagccttggcaaagaaaactggggttggagctggaaggagcgctggattgctgctcggccatgggagattagggttcatgctgagcttcataacctcaagaaagctcatccaaagcaagagagcaaaacgacacttccaaccaaacctgctttgtcaaatgggaaggtgactgctaaatctaaaatgttgcctagcccagctgtggattgtcaagctgcccaggttgccagttctacagccggctcgtctcatctgctgattccaagttga

SEQ ID NO: 2 - protein, 843 aa, Populas trichocarpa nisqually1
MALSMHSKDKQMDSSKYVRYTPEQVEALERVYTECPKPSSLRRQQLIRECPILCNIEPKQIKVWFQNRRCREKQRKESSRLQTVNRKLTAMNKLLMEENDRLQKQVSHLVYENGYMRQQIQTASATATDNSCESVVMSGQHQQQQNPTPQQPQRDANNPAGLLTIAEETLAEFLSKATGTAVDWVQMIGMKPGPDSIGIVAVSRNCSGVAARACGLVSLEPIKVAEILKDRPSWFRDCRCLDIMSVIPTGSGGTIELIYMQTYAPTTLAAARDFWTLRYTTTLEDGSLVICERSLTSSTGGPTGPPSSSFVRADMLPSGYLIRPCEGGGSIIHIVDHVDLDVWSVPEVLRPLYESSKILAQKMTMAALQYIRQIAQETSGEIQYGGGRQPAVLRTFSQRLCRGFNDAVNGFTDSGWSLLDGDGGDDVTIVINSSPTKFLGSQYNASISPTFGGVLCAKASMLLQNVPPALLVRFLREHRSEWADYGVDVYSAACLKASPYAVPCARPGGFPSSQVILPLAHTVEHEEFLEVVRLEGHAFSPEDVALAQDMYLLQLCSGVDENALGACAQLVFAPIDESFADDAPLLPSGFRVIPLDPKTDAPAATRTLDLASTLEVGPGGARPASEADTNSYNLRSVLTIAFQFTFENHFRDNVAAMARQYVRGVVASVQRVAMAISPSRLSSNMGPKSLPVSPEALTLARWIYRSYRIHTGGELFRVDSQAGDALLKRLWHHSDAIMCCSLKTNASPVFTFANQAGLDMLETTLVALQDIMLDKILDEAGRKILCSEFSKIMQQGYAYLPAGICVSSMGRPVSYEQAIAWKVLNDDNSNHCLAFMFINWSFV

SEQ ID NO: 3 - DNA, 2900 bp, Populas trichocarpa nisqually1
cacaatctgggtcatctttgaacttttggttaagcacttgaaaacagtatctgggtggtctttgttgttttcttcaagttttagttctttctttaggtgttttcattgctttacatggcgctttctatgcacagtaaggacaagcagatggattcaagtaagtatgtgcggtatacacctgagcaagttgaggctttggagagggtgtatacagaatgtccaaagcctagttccttgagaagacaacaactcattagagagtgtcctattctctgcaacattgagcctaaacagatcaaagtctggtttcaaaatcgcagatgtcgtgaaaaacagagaaaggaatcttctcgtctccagacagtgaatagaaagctgactgccatgaacaagctgttaatggaagagaatgaccgtttgcagaagcaagtctcccatttggtttatgaaaatggatacatgcgccaacaaattcagaccgcttcagcaacggccacagacaatagctgtgagtctgtggtcatgagcggtcagcaccaacaacagcaaaacccaacacctcagcaaccccaaagggatgctaacaacccagctggtcttctcacaatagctgaggagaccctggcagagttcctttccaaagctactggaactgctgtcgactgggtccagatgattgggatgaagcctggtccagattctattggaatcgttgctgtttcccgcaactgcagtggggtagcagcgcgagcctgtggccttgtgagcctggagcccattaaggttgctgaaatcctcaaagatcgtccttcttggtttcgtgactgccgatgcctagacataatgagtgtgattcctacaggaagtggagggacaattgagctgatatacatgcagacttatgcacctacaacactggccgcagcacgtgacttttggacactaagatacactacaaccttggaagacgggagtcttgtgatatgtgagaggtcattgacttcttctactggtggcccaacaggacctccttcttcaagctttgtaagagctgatatgcttcctagtggctatctaattcgaccatgtgagggtggtggctccatcattcacattgttgatcatgttgacttagatgtttggagtgttcctgaagttctgaggccactttatgagtcatccaaaattcttgctcagaaaatgaccatggcggccttgcagtacataagacaaattgcacaagagactagtggggaaattcagtatggaggtggtcgtcagcctgctgttttaaggacatttagtcagaggctctgcaggggtttcaatgatgctgtaaatggattcacagatagtgggtggtcacttctggatggtgatggtggggatgatgtgaccatcgtcataaattcatcgccaactaaatttctaggatctcaatacaacgcatctatatcacctacatttggaggagtcctgtgtgcgaaggcatcaatgttgctgcagaatgttcctcctgctttgcttgttcgttttctgagagaacatcgttcggagtgggctgattatggggttgatgtgtactctgctgcatgtcttaaagcaagtccttatgcagttccatgtgcaagacctggtggcttccctagtagccaggtcattttacctcttgcacataccgtggagcatgaggagttcttggaggtagttcggctggagggccatgcattctccccagaagatgtagccctggcacaggatatgtacttgttgcagctatgcagcggagttgatgaaaatgctcttggtgcttgtgctcagctcgtctttgcgcctattgatgaatcctttgctgatgatgcaccattgttgccatctggatttcgtgtcataccattagatcctaaaacagatgcacctgctgctactcgaactttagatttagcctctacacttgaagtaggacctggtggtgcccgtccagctagtgaagctgacaccaacagttataaccttaggtcagtactgacaatcgctttccaatttacgtttgagaaccacttccgagacaatgtggctgctatggctcgccaatatgtgcgtggtgttgtggcgtctgttcagagggttgcaatggcaatttccccttcccggctcagctccaatatggggccgaagtcccttcctgtctctcctgaggctttaactttagctcgatggatataccgaagctacaggattcatactggaggagaactatttcgggttgactcccaagctggggatgctttgctgaagcgactgtggcaccattcagatgcaattatgtgctgctccttgaaaacaaacgcatctccagttttcacttttgcaaaccaggctggacttgatatgcttgaaactactcttgtggcccttcaagatatcatgcttgacaagattcttgatgaagcaggtcggaagattctctgctctgagttctctaaaataatgcaacagggatatgcttatctgcccgccggaatatgtgtatccagcatggggaggccggtctcctacgagcaggccattgcatggaaagttcttaatgatgataattccaaccactgtctggctttcatgttcattaactggtcctttgtgtagtggccgggaaagatttttagagagaacttcgctatctattaattagttactatctatctaagtttgtaattttcctcttgctagtacccgtatgactgatgtatggatgcaagtcatggcagaatatcatcaatacttttgtccaaagtgtatgctgacatgttttatggatgttcatgtttctgctgtttatccaagtctatatatcaagttgtcattgtaaatctggtgaaatttcaacaaagagggatgat

SEQ ID NO: 4 - DNA, 30 bp, Artificial Sequence
  Primer
cacccccggggccattgcatggaaagttct

SEQ ID NO: 5 - DNA, 29 bp, Artificial Sequence
  Primer
tctagagatattctgccatgacttgcatc

(19) United States
(12) Patent Application Publication
(10) Pub. No.: US 2019/0136251 A1
(43) Pub. Date: May 9, 2019

(54) GENE IMPACTING CELLULOSE CONTENT AND BIOMASS FORMATION AND METHODS OF USE

(71) Applicant: UT-Battelle, LLC, Oak Ridge, TN (US)

(72) Inventors: Gerald A. Tuskan, Oak Ridge, TN (US); Udaya C. Kalluri, Knoxville, TN (US); Raja S. Payyavula, Henrico, VA (US)

(21) Appl. No.: 15/668,116

(22) Filed: Aug. 3, 2017

Related U.S. Application Data
(60) Provisional application No. 62/370,811, filed on Aug. 4, 2016.

(51) Int. Cl. C12N 15/82; C12Q 1/6895; C12P 7/10

(52) U.S. Cl. CPC C12N 15/8218; C12N 15/8226; C12N 15/8245; C12Q 2600/156; C12P 7/10; C12Q 2600/13; C12Q 1/6895

(57) ABSTRACT

The invention relates to expression of the PdHB3 gene and uses thereof. The invention provides genetically engineered plants expressing the PdHB3 gene and inhibitors of PdHB3. The invention further provides a method for biofuel production using plant genetically engineered for expressing the PdHB3 gene. The invention further provides a method for increasing glucose and/or xylose release in a plant or plant cell.

### CROSS REFERENCE TO RELATED APPLICATIONS

This application claims priority from U.S. Provisional Application No. 62/370,811 filed Aug. 4, 2016, the entire contents of which are incorporated herein by reference.

### STATEMENT REGARDING FEDERALLY SPONSORED RESEARCH

The United States Government has rights in this invention pursuant to contract no. DE-AC05-00OR22725 between the United States Department of Energy and UT-Battelle, LLC. The government has certain rights in this invention.

### FIELD OF THE INVENTION

The invention relates to the field of plant genes. In particular, the invention relates to a gene involved in efficient cellulosic ethanol production.

### INCORPORATION BY REFERENCE OF SEQUENCE LISTING

The Sequence Listing in the ASCII text file, named as ID_3146_1_Sequence_Listing_ST25.txt, created on Aug. 3, 2017, and submitted to the United States Patent and Trademark Office via EFS-Web, is incorporated herein by reference.

### BACKGROUND OF THE INVENTION

Production of renewable fuel from lignocellulosic plant biomass is based on amount, accessibility, extraction and conversion of cellulosic sugars from plant cell wall material. The amount of usable substrate (determined by biomass quantity and cell wall composition) in plant biomass and the ease of its extraction for ethanol conversion purposes are of prime importance in efforts to improve process and cost efficiency of bioethanol production. The ease of extractability of sugar substrates is hampered by plant “recalcitrance”, a term referring to the inherent resistance of plant material to release polysaccharides and other desirable biomaterials from an interwoven matrix of desirable and undesirable materials (Lynd L R. et al., Science 251:1318-1323 (1991)). Extensive thermochemical and enzymatic treatment are needed to overcome this innate recalcitrance of plant stem biomass. Lignin content, its crosslinkages with wall carbohydrates and cell wall architecture are some known causes of recalcitrance. Furthermore, lignin processing creates inhibitory byproducts, such as acetylated compounds, that hamper further extraction and microbe-assisted fermentation. Acetyl esters released during treatment of cell wall polymers can inhibit saccharification of biomass. The released acetate is also inhibitory to the organisms used to ferment the sugars into useful byproducts. Overcoming plant recalcitrance to releasing biomaterials bound in the cell wall as well as increasing the content of desirable substrates (cellulose) are therefore issues of primary importance in the development of biofuel technology.

Cellulose, composed of unbranched chains of beta-1-4 linked D-glucose units, is the most abundant biopolymer on earth and the key substrate for bioethanol production from lignocellulosic biomass. It is suggested in literature that factors such as (i) composition, number and arrangement of CESA (catalytic cellulose biosynthesis enzyme)—containing functional complexes, or rosette complexes and wall associated (KORRIGAN, KOBITO, COBRA) membrane associated (CSI) and soluble proteins (SuSy) influence the cellulose biosynthesis pathway (Endler and Persson, 2011; Mizrachi et al., 2012). Determination of regulatory and signaling pathways critical to rapid turnover of CesAs and CSCs and in turn, the cellulose properties of plant cell walls and biomass are an important consideration in biomass improvement strategies (Kalluri et al. 2014).

Lignins, complex interlinking biopolymers derived from hydroxyphenylpropanoids, provide rigidity and structure to plant cell walls for plant growth and transport of water and nutrients, and are significant contributors to plant recalcitrance. Lignins are composed primarily of syringyl (S), guaiacyl (G) and p-hydroxyphenyl (H) monolignol subunits, which are derived from sinapyl, coniferyl and p-coumaryl alcohols, respectively. The subunit ratio and resulting structure of plant lignins varies according to the genotype, environment, tissue type and maturity of the plant and as such, lignins are very heterogeneous and can vary significantly between different plants, within different tissues of a single plant and even within a single plant cell (Simmons B A et al., Curr Opin Plant Biol. 13:313-20 (2010)). This complexity and heterogeneity hinders the development of conversion technology able to process a range of sustainable feedstocks in a cost-effective manner. A lack of precise understanding of this structure has been an impediment in designing biomass improvement and biomass conversion strategies.

An increase in cellulose and a reduction of lignin content and cell wall recalcitrance, is desirable for biofuel, and pulp and paper industries. Conversely, increases in lignin content of cell walls can be desirable for production of lignin-based products such as carbon fibers, paper and pulp. Thus, genetic manipulation of biomass feedstock to modulate cellulose and lignin biosynthesis, and sugar release efficiency hold promise both for production of improved, economically sustainable lignocellulosic biofuels (Vermerris W. et al., Crop Science 47(S3):S142-S153 (2007); Fu C. et al., PNAS 108:3803-3808 (2011)), and for creating improved cellulose- and lignin-based products.

The genus Populus represents an economically important tree crop that has been targeted for use in diverse applications from the pulp and paper industry, bioremediation, carbon sequestration and as a feedstock in the lignocellulosic biofuel industry (Dinus R J. et al., Crit. Rev. Plant Sci. 20:51-69 (2001)). Recently, a study using wild Populus trichocarpa genotypes collected in the Pacific Northwest region demonstrated high phenotypic variation among the accessions in recalcitrance measured by lignin content and sugar release (Studer M H. et al., PNAS 108:6300-6305 (2011)). This study suggested that sufficient variation occurs in wild germplasm to identify specific genetic determinants of the recalcitrance trait by analysis of naturally-occurring allelic variability.

Quantitative trait loci (QTL) studies have been conducted using interspecific mapping of populations to identify genomic regions associated with cell wall phenotypes linked to recalcitrance (Novaes E. et al., New Phytologist 182:878-890 (2009); Yin T. et al., PLoS one 5:e14021 (2010)). Wegrzyn J L. et al., New Phytologist 188:515-532 (2010) demonstrated the feasibility of using linkage disequilibrium (LD)-based association mapping to validate candidate genes with putative functions in cell wall biosynthesis. The extent of LD decay in P. trichocarpa has been described by Slavov G T. et al., New Phytologist 196(3):713-25 (2012), who reported LD decay to below r²=0.2 within 2 kb in more than half of the genes, within a genomewide average 6-7 kb. Given that the average gene size for P. trichocarpa is 5 kb, these results suggest that QTL fine-mapping and association mapping to within single-gene resolution is possible in P. trichocarpa.

Identification and manipulation of genes regulating cell wall biosynthesis, wood (stem biomass) formation and recalcitrance is critical both for efficient production of cellulosic sugars and ethanol from plant biomass, and for production of improved cellulose-based products, such as paper and pulp and nanocellulose composites.

### SUMMARY OF THE INVENTION

This disclosure provides genetically modified plants characterized by a reduced expression of the PdHB3 gene as compared to a control plant.

The disclosure further provides plants having improved biomass properties of increased quantity and improved conversion to ethanol, methods of generating (genetically modifying) and/or selecting (natural variant collections) plants with such preferred attributes of enhanced growth cellulose content, and ethanol production, and uses of such plants. The inventors have determined that the expression of the gene, Potri.011G098300 (SEQ. ID. NO: 1)(FIG. 1) (designated PdHB3 in Populus deltoides and PtrHB3 in Populus trichocarpa, and predicted to code for a transcription factor protein belonging to the HDZipIII transcription factor family) and/or activity of Potri.011G098300, modulates cellulose, lignin synthesis, sugar release and growth in plants. Plants with improved biomass properties (increased quantity and improved saccharification or conversion efficiency to ethanol), based on the expression or activity of the Potri.011G098300 gene, have divergent uses including pulp and paper production, lignin-based carbon fibers and ethanol/biofuel production.

In one embodiment, methods of generating and/or selecting a plant for improved biomass characteristics are provided. The methods include the steps of (a) obtaining nucleic acids from a candidate plant; (b) DNA construct development or regulating expression of target gene; (c) selecting a plant based on the presence of an allelic variant of the HB3 gene in the nucleic acids obtained from the plant; (d) clarifying phenotypes associated with altered sequence or expression of gene.

Further disclosed herein are nucleic acid inhibitors of expression of Potri.011G098300 gene, or inhibitors of expression of allelic variants of Potri.011G098300 gene (SEQ ID NO: 1), which can be used to reduce expression of the Potri.011G098300 gene and allelic variants thereof, to alter cell wall chemistry and biomass characteristics resulting in improved ethanol production. Specific nucleic acid inhibitors include antisense RNA, small interfering RNA, RNAi, microRNA, artificial microRNA, and ribozymes. Inhibitors of Potri.011G098300 gene activity include vectors or plasmids expressing nucleic acids specific to the target, SEQ ID NO: 1, (for example, an RNAi hairpin loop structure consisting of sense and antisense sequences representative of a gene-specific region in target), operably linked to a regulatory region that is functional in a plant. Also disclosed herein are plants and plant cells genetically modified by introduction of the disclosed inhibitors and expression vectors. Expression of such inhibitors and expression vectors in a plant or plant cell can be used in methods to increase ethanol produced per unit biomass, to increase cellulose content, to increase growth, in such genetically modified plants and plant cells. Further disclosed herein are improved methods of producing biofuel from cellulosic biomass, by using plants with reduced or inhibited expression or activity of the Potri.011G098300 gene (SEQ ID NO: 1) in biofuel production processes.

This disclosure further provides expression vectors with a nucleotide sequence encoding the polypeptide encoded by SEQ ID NO: 1, or another allelic variant of Potri.011G098300 gene, operably linked to a regulatory region that is functional in a plant. The regulatory region can be a promoter with ubiquitous expression, an inducible promoter or a tissue-specific promoter, for example, a xylem-specific promoter. Further provided herein are plants and plant cells genetically modified by introduction of such expression vectors, and methods for improving desirable biomass properties in a plant or plant cell by expressing such expression vectors in a plant or plant cell of interest.

In other embodiments, this disclosure provides methods for increasing glucose and/or xylem release in a plant or plant cell, comprising introducing into said plant or plant cell an expression vector comprising a nucleotide sequence that is transcribed into a nucleic acid inhibitor of expression of the PdHB3 gene operably linked to a regulatory region that is functional in said plant, and expressing the nucleic acid in said plant or plant cell.

In another aspect, this disclosure provides improved methods of producing biofuels comprising using a genetically using a genetically modified plant characterized by a reduced expression of the PdHB3 gene.

Additionally disclosed are methods of producing paper and pulp, by using plants with increased expression of the Potri.011G098300 gene in paper or pulp production processes. Further disclosed are pulp and paper products produced by this method, using plants with increased expression of the Potri.011G098300 gene.

### BRIEF DESCRIPTION OF THE DRAWINGS

FIG. 1: Potri.011G098300 (SEQ. ID NO: 1), Coding sequence (translated region) from reference genome, P. trichocarpa nisqually1.

FIG. 2: Expression (relative abundance) of PtHB3 in different tissues of Populus deltodes. YL—young leaves, ML—mature leaves, YS—young stem, MS—Mature stem, Pet—petiole, Phl—Phloem, Xyl—xylem, Rt—root.

FIG. 3: Full-length protein sequence of PdHB3.

FIGS. 4A-AD: Subcellular localization of PdHB3 protein. Fluorescence from DAPI-stained nucleus as a positive control (A), fluorescence from GFP-tagged PdHB3 (B), overlay of A and B confirming nuclear localization of PdHB3 transcription factor (C), White light image of tobacco mesophyll cells (D).

FIG. 5: Full gene nucleotide sequence (SEQ. ID. NO: 3) of Potri.011G0098300.

FIG. 6: Downregulation of expression level.

FIGS. 7A-7C: PoptrHB3 transic RNAi plants show increased stem volume (A), stem biomass (B), and leaf biomass (C).

FIGS. 8A & 8B: Ethanol conversion (A) and glucose release (B) of PdHB3 transgenic lines.

FIGS. 9A-9C: PoptrHB3 RNAi plants showed altered cell wall composition with higher lignin (A) and cellulose content (B) and S/G ratio (C).

FIG. 10: Related HB3 paralogs.

FIG. 11: Construct design used to develop the HB3 gene-specific RNAi construct.

### DETAILED DESCRIPTION OF THE INVENTION

Disclosed herein are plants having improved biomass properties of increased biomass quantity, increased cellulose content, improved growth, improved ethanol conversion efficiency, methods of generating genetically modifying plants and/or selecting plants with improved biomass properties, and uses of such plants. The inventors have identified a gene, denoted Potri.011 G098300 (SEQ. ID. NO: 1), that modulates plant biomass quantity and quality (i.e., stem biomass quantity/per plant, cellulose content, lignin content and S/G ratio, and sugar release and bioethanol production). Potri.011G0098300 gene, designated PdHB3 in Populus deltoides and PtrHB3 in Populus trichocarpa, encodes a putative transcription factor belonging to the HB3 gene family. HB3 is an eight-membered family in Populus (We et al. 2012). Plants with modulated (increased or decreased) lignin synthesis and sugar release characteristics, based on modulation of the expression or activity of Potri.011G098300 gene, have divergent uses including pulp and paper production, and ethanol/biofuel production.

“Sugar release characteristics” include high or low release of sugars, also referred to as low or high recalcitrance. “High” sugar release (i.e., low recalcitrance) means that sugar can be extracted more easily, or more sugar can be extracted, from a plant, under conditions that would result in less sugar release in a plant without the particular allelic variant. “Low” sugar release (i.e., high recalcitrance) means that sugar can be extracted less easily, or less sugar can be extracted, from a plant, under conditions that would result in more sugar release in a plant without the particular allelic variant. In one example, sugar release refers to the amount of 5- and 6-carbon sugars that can be recovered from a plant using standard techniques to extract these sugars from plant materials. Sugars that can be released include, but are not limited to, glucose, xylose, fructose, arabinose, lactose, ribose, mannose, galactose, and sucrose. Examples of 5-carbon sugars (pentoses) include xylose, ribose, and arabinose; examples of 6-carbon sugars include glucose, fructose, mannose, and galactose.

Sugar release can be measured, for example, by saccharification analysis. In one example of saccharification analysis, sugars are extracted with alpha-amylase and β-glucosidase in sodium acetate, followed by an ethanol soxhlet extraction. After drying overnight, water is added, and samples are sealed and reacted. Once cooled, a buffer-enzyme mix with cellulose oxidative enzymes is added and incubated with the sample. After incubation, an aliquot of the saccharified hydrolysate is tested for sugar content/release, such as by addition of glucose oxidase/peroxidase for measuring glucose content, and/or xylose dehydrogenase to measure xylose content.

Cellulose, a homopolymer of beta 1-4-linked D-glucose units and the most abundant component of secondary cell walls, is synthesized by integral membrane protein complex (CSC) composed on CesA or cellulose synthase proteins. The cellulose properties of quantity, degree of polymerization and crystallinity vary across species, across genotypes of a species and within cell types of an individual plant. Cellulose quantity and quality are significant considerations in selection of feedstocks for cellulosic biofuel or bioproduct production. Cellulose content, crystallinity and degree of polymerization can be affected by modifying the expression of Potri.001G375700 gene in plant.

### Measuring Cellulose in Plants

Cellulose was estimated from air-dried stem (wood) sample using anthrone method (Updegraff, 1969). Stem sample (25 mg) was first digested with 500 μl of acetic—nitric acid reagent (100 ml of 80% acetic acid mixed with 10 ml of nitric acid) at 98° C. for 30 min. After cooling, the sample was centrifuged, discarded the supernatant, and washed with water. After brief centrifugation, water was discarded and the pellet was digested with 67% (v/v) sulfuric acid for 1 hr at room temperature. An aliquot of the mix was diluted (1:10) with water. In a PCR tube, 10 μl of diluted reaction mix, 40 μl of water and 100 μl of freshly prepared anthrone reagent (0.5 mg anthrone ml-1 of cold concentrated sulfuric acid) were added and heated for 10 min at 96° C. Samples were cooled and absorbance (A630) was measured. Cellulose was then estimated based on the absorbance of glucose standards. C6 sugar levels were also estimated by MBMS profiling and wet chemistry methods. Cellulose crystallinity was determined by NMR. Methods of measuring these cellulose properties in biomass are known in the art; see, for example, Foston et al. 2009 and Sluiter A. et al., Determination of structural carbohydrates and lignin in biomass—laboratory analytical procedure. Technical Report NREL/TP-510-42618:1-17 (2008), available from the National Renewable Energy Laboratory.

Lignin” is a complex polymer composed of monolignol subunits, primarily syringyl (S), guaiacyl (G) and p-hydroxyphenyl (H) monolignols, derived from sinapyl, coniferyl and p-coumaryl alcohols, respectively. Lignin forms strong bonds with sugars and interferes with access to these carbohydrates, making it difficult to extract the plant's sugars contained in cellulose and hemicellulose. Differences in lignin content alters the sugar release properties of a plant in the extraction process. Lower lignin levels in a plant are associated with higher levels of sugar release, while higher lignin levels are associated with lower levels of sugar release. Thus, sugar release and lignin content can show an inverse correlation. Lignin content, ratio or amount of monolignols, and expression and/or activity of lignin biosynthetic enzymes, can be affected by modifying the expression of Potri.001G375700 gene in plant.

### Measuring Lignin Synthesis

Methods to determine if a plant has altered lignin synthesis include, for example, directly measuring lignin content, or by determining the expression or activity of genes in the lignin biosynthetic pathway. Lignin content can be measured directly, for example, by thioglycolysis, or by histochemical analysis of tissue sections stained with toluidine blue O (TBO), Wiesner reagent, or Maule reagent to identify lignified or non-lignified cell walls. Liginin may also be measured by pyrolysis vapor analysis using pyrolysis Molecular Beam Mass Spectrometry (py-MBMS) (Evans R J. et al., Energy and Fuels 1:123-137 (1987); Sykes R. et al., Biofuels: Methods and Protocols 169-183 (2009); Tuskan G. et al., Appl. Biochem. Biotechnol. 77:55-65 (1999)). Additional methods of measuring carbohydrate and lignin content in biomass are known in the art; see, for example, Sluiter A. et al., Determination of structural carbohydrates and lignin in biomass—laboratory analytical procedure. Technical Report NREL/TP-510-42618:1-17 (2008), available from the National Renewable Energy Laboratory.

Methods to Select Plants with Improved Biomass Properties.

In one embodiment, methods of generating and selecting a plant for improved biomass properties of increased biomass quantity, increased cell wall sugar content and deceased lignin content and improved sugar release efficiency are provided. The methods include the steps of (a) obtaining nucleic acids from a candidate plant; (b) design, construction, delivery and verification of integration in host plant (Populus deltoides) genome of a RNAi gene down-regulation construct for Potri.011G098300 target gene; and (c) selection of plants (carrying RNAi construct of Potri.011G098300) with improved biomass properties based as determined by quantitative analysis of stem biomass, cellulose and lignin content, and sugar release and/or ethanol production using existing art.

The first step in generating and selecting a plant with modified expression of target gene Potri.011G098300 is to obtain (a) experimental evidence (gene expression context) in support of potential molecular role and secondly, (b) validating the functional role (via RNAi transgene technology and SNP association analysis) in impacting biomass properties nucleic acids from a candidate plant. Experimental evidence in support of a potential molecular role for the target gene Potri.0110098300, belonging to the HB3 gene family, described here was originally identified by the inventors as highly expressed in developing xylem tissue, especially under tension stress. Co-expression analyses of in-house and publicly available datasets confirmed the expression context during biomass formation and suggesting a potential role. Validation of the functional role was undertaken by generating RNAi plants with down regulated levels of target gene expression Potri.011G6098300 and detailed characterization of such plants for changes in biomass properties.

Methods of obtaining nucleic acids from a candidate plant, detecting the presence of a nucleotide sequence and constructing DNA clones (enzyme based amplification, sequencing, digestion, ligation and recombination) are known in the art. Nucleic acid can be isolated from a plant tissue sample, according to standard methodologies (Sambrook et al., Molecular Cloning, 2nd ed., Cold Spring Harbor Laboratory Press, CSH, 1.38-1.39, 1989).

A number of template dependent processes are available to amplify the marker sequences present in a given nucleic acid sample. One of the best known amplification methods is the polymerase chain reaction (referred to as PCR). Other methods of amplification are ligase chain reaction (LCR), Qbeta Replicase, isothermal amplification, strand displacement amplification (SDA), PCR-like template- and enzyme-dependent synthesis using primers with a capture or detector moiety, transcription-based amplification systems (TAS), cyclical synthesis of single-stranded and double-stranded DNA, “RACE”, one-sided PCR, and di-oligonucleotide amplification.

The PCR method is well known in the art and disclosed, for example, in WO 99/28500; Sambrook et al. (Molecular Cloning—A Laboratory Manual, Cold Spring Harbor Laboratory, Cold Spring Harbor, N.Y. 1989); Nucleic Acid Hybridization (Hames and Higgins eds., 1984); and Current Protocols in Human Genetics (Dracopoli et al., eds, 1984 with quarterly updates, John Wiley & Sons, Inc.), all of which are incorporated herein by reference. The PCR method utilizes a pair of oligonucleotide primers, each hybridizing to one strand of a double-stranded DNA/RNA target. The primers flank the region that will be amplified. The PCR method comprises contacting the primers and target sequence, or mixture of target sequences and optional polynucleotide probes, and performing the amplification steps.

Primers for nucleic acid amplification of the Potri.011G098300 gene should contain a hybridizing region exactly or substantially complementary or corresponding to a target nucleotide sequence. Primer extension is performed under hybridization conditions of sufficient stringency to allow the selective amplification of the target sequence. A primer can either consist entirely of the hybridizing region or can contain additional features which allow for detection, immobilization, or manipulation of the amplified product, but which do not alter the basic property of the primer (that is, acting as a point of initiation of DNA synthesis).

The generation of plants with altered activity of the gene(s) orthologous to the gene Potri.011G098300 in other crops species and/or selection of missense allelic variants of the gene(s) orthologous to the Potri.01l 1098300 gene in other crop species allows for selection and screening of candidate plants for having improved sugar release or biomass formation characteristics. These biomass characteristics include a combination of high or low sugar release, such as high or low release of glucose and/or xylose, high or low biomass quantity or density and high or low cellulose or lignin levels. Preferred characteristics include, high biomass and high release of glucose and/or xylose.

### Selection and Screening Using the Potri.011G09S300 Gene

The gene Potri.011G098300 expression can be modified using transgenic technology or sequence of an allelic variant of the Potri.011G098300 gene resulting in decreased gene activity, can be used as a molecular marker for use in screening germplasm in plant breeding and genetic improvement efforts.

### Inhibitors and Expression Vectors for Modulating the Activity of Potri.011G098300

Disclosed herein are nucleic acid inhibitors of expression of Potri.011G098300, or inhibitors of expression of allelic variants of Potri.011G098300, which can be used to reduce expression of the Potri.011G098300 gene and allelic variants thereof, to provide low lignin biosynthesis and high sugar release. Specific nucleic acid inhibitors include antisense RNA, small interfering RNA, RNAi, microRNA, artificial microRNA, and ribozymes. Inhibitors of Potri.011G098300 activity include expression vectors encoding the polypeptide of SEQ ID NO: 1, operably linked to a regulatory region that is functional in a plant.

The polynucleotides and expression vectors described herein can be used to increase or inhibit expression of Potri.011G098300 or a Potri.011G098300 allelic variant. The term “expression” refers to the process of converting genetic information of a polynucleotide into RNA through transcription, which is catalyzed by an enzyme, RNA polymerase and into protein, through translation of mRNA on ribosomes. Up-regulation or overexpression refers to regulation that increases the production of expression products (mRNA, polypeptide or both) relative to basal or native states, while inhibition or down-regulation refers to regulation that decreases production of expression products (mRNA, polypeptide or both) relative to basal or native states.

A “nucleic acid inhibitor” is a nucleic acid that can reduce or prevent expression or activity of a target gene. For example, an inhibitor of expression of Potri.011G098300 can reduce or eliminate transcription and/or translation of the Potri.011G098300 gene product, thus reducing Potri.011G098300 protein expression.

The term “control plant” as used herein refers to a plant cell, an explant, seed, plant component, plant tissue, plant organ, or whole plant used to compare against transgenic or genetically modified plant for the purpose of identifying an enhanced phenotype or a desirable trait in the transgenic or genetically modified plant. A “control plant” may in some cases be a transgenic plant line that comprises an empty vector or marker gene, but does not contain the recombinant polynucleotide of interest that is present in the transgenic or genetically modified plant being evaluated. A control plant may be a plant of the same line or variety as the transgenic or genetically modified plant being tested, or it may be another line or variety, such as a plant known to have a specific phenotype, characteristic, or known genotype. A suitable control plant would include a genetically unaltered or non-transgenic plant of the parental line used to generate a transgenic plant herein

An altered level of gene expression refers to a measurable or observable change in the level of expression of a transcript of a gene, or the amount of its corresponding polypeptide, relative to a control plant or plant cell under the same conditions (e.g., as measured through a suitable assay such as quantitative RT-PCR, a Northern blot, a Western blot or through an observable change in phenotype, chemical profile or metabolic profile). An altered level of gene expression can include up-regulated or down-regulated expression of a transcript of a gene or polypeptide relative to a control plant or plant cell under the same conditions. Altered expression levels can occur under different environmental or developmental conditions or in different locations than those exhibited by a plant or plant cell in its native state.

Techniques for introducing nucleic acids (inhibitors and expression vectors) into monocotyledonous and dicotyledonous plants are known in the art and include, without limitation, Agrobacterium-mediated transformation, viral vector-mediated transformation, electroporation and particle gun transformation, e.g., U.S. Pat. Nos. 5,538,880, 5,204,253, 6,329,571 and 6,013,863. If a cell or tissue culture is used as the recipient tissue for transformation, plants can be regenerated from transformed cultures if desired, by techniques known to those skilled in the art. See, e.g., Niu et al., Plant Cell Rep. V19:304-310 (2000); Chang and Yang, Bot. Bull. Acad Sin., V37:35-40 (1996) and Han et al., Biotechnology in Agriculture and Forestry, V44:291 (ed. by Y. P. S. Bajaj), Springer-Vernag, (1999).

### Nucleic Acid Inhibitors

A number of nucleic acid based methods, including antisense RNA, ribozyme directed RNA cleavage, post-transcriptional gene silencing (PTGS), e.g., RNA interference (RNAi), microRNA and artificial microRNA and transcriptional gene silencing (TGS) can be used to inhibit Potri.011G098300 expression in plants. Suitable inhibitors include full-length nucleic acids of allelic variants of Potri.011G098300, or fragments of such full-length nucleic acids. In some embodiments, a complement of the full-length nucleic acid or a fragment thereof can be used. Typically, a fragment is at least 10 nucleotides, e.g., at least 12, 13, 14, 15, 16, 17, 18, 19, 20, 21, 22, 23, 24, 25, 26, 27, 30, 35, 40, 50, 80, 100, 200, 500 nucleotides or more. Generally, higher homology can be used to compensate for the use of a shorter sequence.

Antisense technology is one well-known method. In this method, a nucleic acid segment from a gene to be repressed is cloned and operably linked to a regulatory region and a transcription termination sequence so that the antisense strand of RNA is transcribed. The recombinant vector is then transformed into plants, as described below and the antisense strand of RNA is produced. The nucleic acid segment need not be the entire sequence of the gene to be repressed, but typically will be substantially complementary to at least a portion of the sense strand of the gene to be repressed.

In another method, a nucleic acid can be transcribed into a ribozyme or catalytic RNA, which affects expression of an mRNA. See, U.S. Pat. No. 6,423,885. Ribozymes can be designed to specifically pair with a target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes cleave mRNAs at locations dictated by flanking regions that form complementary base pairs with the target mRNA. See, for example, U.S. Pat. No. 5,254,678; Perriman et al., PNAS 92(13):6175-6179 (1995); de Feyter and Gaudron, Methods in Molecular Biology, Vol. 74, Chapter 43, Edited by Turner, P. C., Humana Press Inc., Totowa, N.J. RNA endoribonucleases which have been described, such as the one that occurs naturally in Tetrahymena thermophila, can be useful. See, for example, U.S. Pat. Nos. 4,987,071 and 6,423,885.

PTGS, e.g., RNAi, can also be used to inhibit the expression of a gene. For example, a construct can be prepared that includes a sequence that is transcribed into an RNA that can anneal to itself, e.g., a double stranded RNA having a stem-loop structure. In some embodiments, one strand of the stem portion of a double stranded RNA comprises a sequence that is similar or identical to the sense coding sequence or a fragment thereof, of the polypeptide of interest. The length of the sequence that is similar or identical to the sense coding sequence can be from 10 nucleotides to 500 nucleotides, from 15 nucleotides to 300 nucleotides, from 20 nucleotides to 100 nucleotides or from 25 nucleotides to 100 nucleotides. The other strand of the stem portion of a double stranded RNA comprises a sequence that is similar or identical to the antisense strand or a fragment thereof, of the coding sequence of the polypeptide of interest and can have a length that is shorter, the same as or longer than the corresponding length of the sense sequence. In some cases, one strand of the stem portion of a double stranded RNA comprises a sequence that is similar or identical to the 3′ or 5′ untranslated region or a fragment thereof, of the mRNA encoding the polypeptide of interest and the other strand of the stem portion of the double stranded RNA comprises a sequence that is similar or identical to the sequence that is complementary to the 3′ or 5′ untranslated region, respectively or a fragment thereof, of the mRNA encoding the polypeptide of interest. In other embodiments, one strand of the stem portion of a double stranded RNA comprises a sequence that is similar or identical to the sequence of an intron or a fragment thereof in the pre-mRNA encoding the polypeptide of interest and the other strand of the stem portion comprises a sequence that is similar or identical to the sequence that is complementary to the sequence of the intron or fragment thereof in the pre-mRNA.

A construct including a sequence that is operably linked to a regulatory region and a transcription termination sequence and that is transcribed into an RNA that can form a double stranded RNA, can be transformed into plants as described below. Methods for using RNAi to inhibit the expression of a gene are known to those of skill in the art. See, e.g., U.S. Pat. Nos. 5,034,323; 6,326,527; 6,452,067; 6,573,099; 6,753,139; and 6,777,588. See also WO 97/01952; WO 98/53083; WO 99/32619; WO 98/36083; and U.S. Patent Publications 20030175965, 20030175783, 20040214330 and 20030180945.

In some embodiments, a construct containing a nucleic acid having at least one strand that is a template for both sense and antisense sequences that are complementary to each other is used to inhibit the expression of a gene. The sense and antisense sequences can be part of a larger nucleic acid molecule or can be part of separate nucleic acid molecules having sequences that are not complementary. The sense or antisense sequence can be a sequence that is identical or complementary to the sequence of an mRNA, the 3′ or 5′ untranslated region of an mRNA or an intron in a pre-mRNA encoding a polypeptide of interest or a fragment of such sequences. In some embodiments, the sense or antisense sequence is identical or complementary to a sequence of the regulatory region that drives transcription of the gene encoding a polypeptide of interest. In each case, the sense sequence is the sequence that is complementary to the antisense sequence.

A nucleic acid having at least one strand that is a template for one or more sense and/or antisense sequences can be operably linked to a regulatory region to drive transcription of an RNA molecule containing the sense and/or antisense sequence(s). In addition, such a nucleic acid can be operably linked to a transcription terminator sequence, such as the terminator of the nopaline synthase (nos) gene. In some cases, two regulatory regions can direct transcription of two transcripts: one from the top strand and one from the bottom strand. See, for example, Yan et al., Plant Physiol., 141:1508-1518 (2006). The two regulatory regions can be the same or different. The two transcripts can form double-stranded RNA molecules that induce degradation of the target RNA. In some cases, a nucleic acid can be positioned within a P-DNA such that the left and right border-like sequences of the P-DNA are on either side of the nucleic acid.

In some embodiments, a suitable nucleic acid inhibitor can be a nucleic acid analog. Nucleic acid analogs can be modified at the base moiety, sugar moiety or phosphate backbone to improve, for example, stability, hybridization or solubility of the nucleic acid. Modifications at the base moiety include deoxyuridine for deoxythymidine and 5-methyl-2′-deoxycytidine and 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered morpholino ring or peptide nucleic acids, in which the deoxyphosphate backbone is replaced by a pseudopeptide backbone and the four bases are retained. See, for example, Summerton and Weller, 1997, Antisense Nucleic Acid Drug Dev., 7:187-195; Hyrup et al., Bioorgan. Med. Chem., 4:5-23 (1996). In addition, the deoxyphosphate backbone can be replaced with, for example, a phosphorothioate or phosphorodithioate backbone, a phosphoroamidite or an alkyl phosphotriester backbone.

### Expression Vector Modulators of Potri.011G098300 and Uses Thereof.

This disclosure provides methods of altering biomass properties, specifically, increasing biomass quantity and density, increasing cellulose content and decreasing lignin content and recalcitrance, in a plant, comprising introducing into a plant cell an exogenous nucleic acid with a regulatory region operably linked to a nucleic acid encoding a Potri.011G098300 allelic variant, where a tissue of a plant produced from the plant cell has an altered cell wall compared to the cell wall in tissue of a control plant that does not comprise the nucleic acid.

Vectors containing nucleic acids such as those described herein are provided. A “vector” is a replicon, such as a plasmid, phage or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs or PACs. The term “vector” includes cloning and expression vectors, as well as viral vectors and integrating vectors. An “expression vector” is a vector that includes a regulatory region. Suitable expression vectors include, without limitation, plasmids and viral vectors derived from, for example, bacteriophage, baculoviruses and retroviruses. Numerous vectors and expression systems are commercially available from such corporations as Novagen (Madison, Wis.), Clontech (Mountain View, Calif.), Stratagene (La Jolla, Calif.) and Invitrogen/Life Technologies (Carlsbad, Calif.).

The vectors provided herein also can include, for example origins of replication, scaffold attachment regions (SARs) and/or markers. A marker gene can confer a selectable phenotype on a plant cell. For example, a marker can confer biocide resistance, such as resistance to an antibiotic (e.g., kanamycin, G418, bleomycin or hygromycin) or an herbicide (e.g., chlorosulfuron or phosphinothricin). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin or Flag-tag (Kodak, New Haven, Conn.) sequences typically are expressed as a fusion with the encoded polypeptide. Such tags can be inserted anywhere within the polypeptide, including at either the carboxyl or amino terminus. As described herein, plant cells can be transformed with a recombinant nucleic acid construct to express a polypeptide of interest.

The term “regulatory region” refers to a nucleic acid having nucleotide sequences that influence transcription or translation initiation and rate and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns and combinations thereof.

The term “operably linked” refers to positioning of a regulatory region and a sequence to be transcribed in a nucleic acid so as to influence transcription or translation of such a sequence. For example, to bring a coding sequence under the control of a regulatory region, the translation initiation site of the translational reading frame of the polypeptide is typically positioned between one and about fifty nucleotides downstream of the promoter. A regulatory region can, however, be positioned as much as about 5,000 nucleotides upstream of the translation initiation site or about 2,000 nucleotides upstream of the transcription start site. A regulatory region typically comprises at least a core (basal) promoter.

A regulatory region also may include at least one control element, such as an enhancer sequence, an upstream element or an upstream activation region (UAR). For example, a suitable enhancer is a cis-regulatory element (−212 to −154) from the upstream region of the octopine synthase (ocs) gene (Fromm et al., The Plant Cell 1:977-984 (1989)). The choice of regulatory regions to be included depends upon several factors, including, but not limited to, efficiency, selectability, inducibility, desired expression level and cell- or tissue-preferential expression. It is a routine matter for one of skill in the art to modulate the expression of a coding sequence by appropriately selecting and positioning regulatory regions relative to the coding sequence.

A variety of promoters are available for use, depending on the degree of expression desired. For example, a broadly expressing promoter promotes transcription in many, but not necessarily all, plant tissues. Non-limiting examples of broadly expressing promoters that can be included in the nucleic acid constructs provided herein include the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter and ubiquitin promoters such as the maize ubiquitin-1 promoter.

Some suitable regulatory regions initiate transcription, only or predominantly, in certain cell types. For example, a promoter that is active predominantly in a reproductive tissue (e.g., fruit, ovule or inflorescence) can be used. Thus, as used herein a cell type- or tissue-preferential promoter is one that drives expression preferentially in the target tissue, but may also lead to some expression in other cell types or tissues as well.

Root-active and root-preferential promoters confer transcription in root tissue, e.g., root endodermis, root epidermis or root vascular tissues. Root-preferential promoters include the root-specific subdomains of the CaMV 35S promoter (Lam et al., Proc. Natl. Acad Sci. USA, 86:7890-7894 (1989)), root cell specific promoters reported by Conkling et al., Plant Physiol., 93:1203-1211 (1990) and the tobacco RD2 promoter.

Promoters active in photosynthetic tissue confer transcription in green tissues such as leaves and stems. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol., 35:773-778 (1994)), the Cab-1 promoter from wheat (Fejes et al., Plant Mol. Biol., 15:921-932 (1990)), the CAB-1 promoter from spinach (Lubberstedt et al., Plant Physiol., 104:997-1006 (1994)), the cab IR promoter from rice (Luan et al., Plant Cell, 4:971-981 (1992)), the pyruvate orthophosphate dikinase (PPDK) promoter from corn (Matsuoka et al., Proc. Natl. Acad Sci. USA, 90:9586-9590 (1993)), the tobacco Lhcb1*2 promoter (Cerdan et al., Plant Mol. Biol., 33:245-255 (1997)), the Arabidopsis SUC2 sucrose-H+ symporter promoter (Truernit et al., Planta, 196:564-570 (1995)) and thylakoid membrane protein promoters from spinach (psaD, psaF, psaE, PC, FNR, atpC, atpD, cab, rbcS).

Lignin biosynthesis promoters are promoters that drive transcription of nucleic acids encoding enzymes involved in lignin biosynthesis. Examples of lignin biosynthesis promoters include promoters of the switchgrass (Panicum virgatum), rice (Oryza sativa), corn (Zea mays) and wheat (Triticum aestivum) homologs of the Populus cinnamate 4-hydroxylase, caffeoyl-CoA O-methyltransferase and caffeic acid O-methyltransferase genes. Also suitable are promoters of Arabidopsis genes encoding phenylalanin ammonia lyase (genomic locus At3g10340), trans-cinnamate 4-hydroxylase (genomic locus At2g30490), 4-coumarate:CoA ligase (genomic locus At1g51680), hydroxycinnamoyl-CoA:shikimate/quinate hydroxycinnamoyltransferase (genomic locus At5g48930), p-coumarate 3-hydroxylase (genomic locus At2g40890), caffeoyl-CoA 3-O-methyltransferase (genomic locus At4g34050), cinnamoyl CoA reductase (genomic locus At1g15950), ferulate 5-hydroxylase (genomic locus At4g36220), caffeic acid O-methyltransferase (genomic locus At5g54160) and cinnamyl alcohol dehydrogenase (genomic locus At4g34230).

Useful promoters also include cell wall related promoters, such as cellulose biosynthesis promoters. Cellulose biosynthesis promoters are promoters that drive transcription of nucleic acids encoding enzymes involved in cellulose biosynthesis. Examples of cellulose biosynthesis promoters include the promoter of the rice cellulose synthase gene (genomic locus Os08g25710), the promoter of the rice cellulose synthase gene (genomic locus Os08g06380) and the promoter of the rice cellulose synthase-like A2 gene (genomic locus Os10g26630).

Examples of promoters that have high or preferential activity in vascular bundles include the glycine-rich cell wall protein GRP 1.8 promoter (Keller and Baumgartner, Plant Cell, 3(10):1051-1061 (1991)), the Commelina yellow mottle virus (CoYMV) promoter (Medberry et al., Plant Cell, 4(2):185-192 (1992)) and the rice tungro bacilliform virus (RTBV) promoter (Dai et al., Proc. Natl. Acad. Sci. USA, 101(2):687-692 (2004)). Promoters having preferential activity in the phloem region (e.g., primary phloem cells, companion cells and sieve cells), the xylem region (e.g., tracheids and vessels), the bundle sheath layer and/or the endodermis are also considered vascular tissue promoters. Promoters that have preferential activity in the pith, cortex, epidermis and/or in the vascular bundles or vascular layers of the stem are considered stem promoters. In some cases, the activity of stem promoters can also be induced by stress like drought.

Inducible promoters confer transcription in response to external stimuli such as chemical agents or environmental stimuli. For example, inducible promoters can confer transcription in response to hormones such as gibberellic acid or ethylene or in response to light, nitrogen, shade or drought.

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

A 5′ untranslated region (UTR) can be included in nucleic acid constructs described herein. A 5′ UTR is transcribed, but is not translated and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a recombinant polynucleotide, e.g., introns, enhancers, upstream activation regions, transcription terminators and inducible elements. Thus, for example, more than one regulatory region can be operably linked to the sequence of a polynucleotide encoding a Gene Y homolog or other lignin-modulating polypeptide. Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

In one example, the coding sequence of gene Potri.011G098300 (SEQ ID NO: 1) is amplified from either genomic DNA or cDNA by PCR. The DNA fragments are then subcloned into an expression construct.

### Transgenic Plants/Plant Species/Plant Cells

Also disclosed herein are plants and plant cells genetically modified by introduction of the disclosed inhibitors and expression vectors including nucleic acids encoding a specific Hairpin-loop fragments of one gene Potri.011G098300.

A plant or plant cell used in methods of the invention contains a recombinant nucleic acid construct as described herein. A plant or plant cell can be transformed by having a construct integrated into its genome, i.e., can be stably transformed. Stably transformed cells typically retain the introduced nucleic acid with each cell division. A plant or plant cell can also be transiently transformed such that the construct is not integrated into its genome. Transiently transformed cells typically lose all or some portion of the introduced nucleic acid construct with each cell division such that the introduced nucleic acid cannot be detected in daughter cells after a sufficient number of cell divisions. Both transiently transformed and stably transformed transgenic plants and plant cells can be useful in the methods described herein.

Typically, transgenic plant cells used in methods described herein constitute part or all of a whole plant. Such plants can be grown in a manner suitable for the species under consideration, via propagation of seeds or vegetative cuttings (as in case of Populus) in a growth chamber, a greenhouse or in a field. Transgenic plants can be bred as desired for a particular purpose, e.g., to introduce a recombinant nucleic acid into other lines, to transfer a recombinant nucleic acid to other species or for further selection of other desirable traits. Progeny includes descendants of a particular plant or plant line provided the progeny inherits the transgene. Progeny of a plant include seeds formed on F1, F2, F3, F4, F5, F6 and subsequent generation plants or seeds formed on BC1, BC2, BC3 and subsequent generation plants or seeds formed on F1BC1, F1BC2, F1BC3 and subsequent generation plants. Seeds produced by a transgenic plant can be grown and then selfed (or outcrossed and selfed) to obtain seeds homozygous for the nucleic acid construct. Alternatively, transgenic plants can be propagated vegetatively for those species amenable to such techniques.

Transgenic plant cells growing in suspension culture or tissue or organ culture can be useful for extraction of polypeptides or compounds of interest, e.g., lignin monomers or compounds in a lignin biosynthetic pathway. For the purposes of this invention, solid and/or liquid tissue culture techniques can be used. When using solid medium, transgenic plant cells can be placed directly onto the medium or can be placed onto a filter film that is then placed in contact with the medium. When using liquid medium, transgenic plant cells can be placed onto a floatation device, e.g., a porous membrane that contacts the liquid medium. Solid medium typically is made from liquid medium by adding agar. For example, a solid medium can be any of various mineral salt media, e.g., Murashige and Skoog (MS) medium containing agar and a suitable concentration of an auxin, e.g., 2,4-dichlorophenoxyacetic acid (2,4-D) and a suitable concentration of a cytokinin, e.g., kinetin.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days or about 1-3 days. The use of transient assays is particularly convenient for rapid analysis in different species or to confirm expression of a heterologous Potri.011G098300 whose expression, subcellular localization and protein interaction partner has not previously been confirmed in particular recipient cells.

Initial and immediate application of the expression of Potri.011G098300 allelic variants can be made in the bioenergy crops Populus and switchgrass, but the application can be extended to other bioenergy crops such as corn, other sources of lignocellulosic biomass and other model plants e.g., Salix, Miscanthus, rice and Medicago.

For example, the polynucleotides and vectors described herein can be used to transform a number of monocotyledonous and dicotyledonous plants and plant cell systems, including alfalfa, ash, beech, birch, canola, cherry, clover, cotton, cottonseed, eucalyptus, flax, jatropha, mahogany, maple, mustard, oak, poplar, oilseed rape, rapeseed (high erucic acid and canola), red clover, teak, tomato, walnut and willow, as well as monocots such as barley, bluegrass, canarygrass, corn, fescue, field corn, millet, miscanthus, oat, rice, rye, ryegrass, sorghum, sudangrass, sugarcane, sweet corn, switchgrass, turf grasses, timothy and wheat Gymnosperms such as fir, pine and spruce can also be suitable.

The methods and compositions can be used over a broad range of plant species, including species from the dicot genera Acer, Afzelia, Arabidopsis, Betula, Brassica, Eucalyptus, Fagus, Fraxinus, Glycine, Gossypium, Jatropha, Juglans, Linum, Lycopersicon, Medicago, Micropus, Populus, Prunus, Quercus, Salix, Solanum, Tectona and Trifolium; and the monocot genera Agrostis, Avena, Festuca, Hordeum, Lemna, Lolium, Milium, Miscanthus oryza, Panicum, Pennisetum, Phalaris, Phleum, Poa, Saccharum, Secale, Sorghum, Triticum, Zea and Zoysia; and the gymnosperm genera Abies, Picea and Pinus. In some embodiments, a plant is a member of the species Festuca arundinacea, Miscanthus hybrid (Miscanthus×giganteus), Miscanthus sinensis, Miscanthus sacchariflorus, Panicum virgatum, Pennisetum purpureum, Phalaris arundinacea, Populus spp including but not limited to balsamifera, deltoides, tremuloides, tremula, alba and maximowiczil, Saccharum spp., Secale cereale, Sorghum almum, Sorghum halcapense or Sorghum vulgare. In certain embodiments, the polynucleotides and vectors described herein can be used to transform a number of monocotyledonous and dicotyledonous plants and plant cell systems, wherein such plants are hybrids of different species.

In another aspect, a plant cell comprising a Potri.011G098300 nucleic acid inhibitor is provided. The plant cell comprises an exogenous nucleic acid, the exogenous nucleic acid comprising a regulatory region operably linked to a polynucleotide that is transcribed into an interfering RNA effective for inhibiting expression of Potri.011G098300. The exogenous nucleic acid can further comprise a 3′ UTR operably linked to the polynucleotide. The polynucleotide can be transcribed into an interfering RNA comprising a stem-loop structure. The stem-loop structure can comprise an inverted repeat of the 3′ UTR.

In another aspect, a plant is provided. The plant comprises any of the plant cells described above. Progeny of the plant also are provided, where the progeny have altered (increased or decreased) cellulose and lignin content.

### Methods of Use of Transgenic Plants

Disclosed herein are methods to increase glucose and/or xylose release in a plant or plant cell, increase biomass quantity or density, increase cellulose biosynthesis or to decrease lignin synthesis, by expressing the disclosed inhibitors in plants and plant cells.

Further disclosed herein are improved methods of producing biofuel from cellulosic biomass, by using plants with reduced or inhibited expression or activity of the Potri.011G098300 gene in biofuel production processes. Methods of pretreatment and saccharification of biomass to fermentable sugars, followed by fermentation of the sugars to ethanol, are known in the art.

The term “biofuel” refers to any type of fuel which is derived in any way from biomass. In some embodiments, the biofuel in the context of the present invention is a liquid biofuel. The biofuel may mainly comprise an extensively pure compound, thus, may be a biofuel comprising more than 95% of said compound and less than 5% of one or more other compound(s), of more than 80% of said compound and less than 20% of one or more other compound(s) or of more than 75% of said compound and less than 25% of one or more other compound(s). Alternatively, the biofuel may be a mixture of different compounds.

In some embodiments, the biofuel comprises one or more alcohol(s), one or more ester(s), one or more carbonic acid(s), one or more ketone(s), one or more aldehyde(s) or one and/or more terpene(s). In some embodiments, the biofuel comprises one or more alcohol(s), one or more ketone(s) (e.g., acetone), one or more aldehyde(s) and/or comprises one or more ester(s). In some embodiments, the biofuel comprises one or more alcohol(s) and/or comprises one or more ester(s). In some embodiments, the biofuel may comprise more than 50% (v/v), more than 70% (v/v), more than 80% (v/v), more than 90% (v/v) or more than 95% (v/v) of one or more alcohol(s). In some embodiments, these alcohols are aliphatic alcohols (e.g., methanol, ethanol, n-propanol, isopropanol and/or butanol), specifically aliphatic alcohols of the general molecular formula H—CₙH₂ₙ—OH, even more specifically, one of the first four aliphatic alcohols with n=1-4 (i.e., methanol, ethanol, propanol and/or butanol). In the context of the present invention these alcohols may also be designated as “bioalcohols” (i.e., as “biomethanol”, “bioethanol”, “biopropanol” and “biobutanol”). Due to its chemical and technical characteristics, in the context of biofuel, butanol is sometimes also designated as “biogasoline”. In some embodiments, the alcohol may be a di-, tri or polyalcohol such as, e.g., glycerol. In some embodiments, the biofuel in the context of the present invention comprises more than 50% (v/v), more than 70% (v/v), more than 80% (v/v), more than 90% (v/v), or more than 95% (v/v) ethanol. In a specific embodiment, the biofuel of the present invention comprises at least 90% (v/v) ethanol.

### Articles of Manufacture

The materials and methods described herein are useful for modifying biomass characteristics, such as characteristics of biomass renewable energy source plants. “Biomass” refers to any cellulosic or lignocellulosic material and includes materials containing cellulose, and optionally further containing hemicellulose, lignin, starch, oligosaccharides and/or monosaccharides. The term “cellulosic” refers to a composition containing cellulose. The term “lignocellulosic” refers to a composition containing both lignin and cellulose. According to the invention, biomass may be derived from a single source, or biomass can contain a mixture derived from more than one source; for example, biomass can contain a mixture of corn cobs and corn stover, or a mixture of grass and leaves. Examples of biomass include, but are not limited to, tree crops such as Populus, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from processing of grains, trees, branches, roots, leaves, wood chips, sawdust, shrubs and bushes, vegetables, and fruits.

Lignin itself, which can be gathered from transgenic plants provided herein, can be converted into valuable fuel additives. Lignin can be recovered from any bioethanol production process using agricultural materials such as straw, corn stalks and switchgrass engineered to have increased lignin content. Lignin can be combusted to provide heat and/or power for the ethanol process; however, increasing the value of the lignin by converting it to higher value fuel additives can significantly enhance the competitiveness of bioethanol technology. Lignins removed from wood pulp as sulphates can be used as dust suppression agents for roads, as dispersants in high performance cement applications, water treatment formulations and textile dyes or as raw materials for several chemicals, such as vanillin, DMSA, ethanol, torula yeast, xylitol sugar and humic acid.

The invention also relates to the use of the pulp obtained from the disclosed genetically modified plants in the production of cellulose-based products, for example, in the paper industry, or for the production of cardboard. Pulp, produced using plants which have been genetically modified to have increased expression of the Potri.011G098300 gene and therefore also have increased lignin synthesis, can be used as a building material and in particular as output material for pressed chipboard, fiberboard of medium density, or as filler material.

Seeds of transgenic plants described herein can be conditioned and bagged in packaging material by means known in the art to form an article of manufacture. Packaging material such as paper and cloth are well known in the art. A package of seed can have a label, e.g., a tag or label secured to the packaging material, a label printed on the packaging material or a label inserted within the package. The package label may indicate that the seed herein incorporates transgenes that provide increased amounts of lignin or altered S/G lignin ratio in one or more tissues of plants grown from such seeds.

Further disclosed herein are methods for identifying suitable allelic variants, primers targeting conserved regions of the gene can be used to identify genotypes carrying alterations that lead to amino acid substitutions which can affect gene function.

A population of plants can be screened or selected for those members of the population that have a desired trait or phenotype. Selection or screening can be carried out over one or more generations, which can be useful to identify those plants that have a desired characteristic, such as high biomass content with low recalcitrance, low lignin synthesis, and/or high sugar release. Selection or screening can be carried out in more than one geographic location. In some cases, transgenic plants can be grown and selected under conditions which induce a desired phenotype or are otherwise necessary to produce a desired phenotype in a transgenic plant. In addition, selection or screening can be carried out during a particular developmental stage in which the phenotype is exhibited by the plant.

The present disclosure is further illustrated by the following non-limiting examples.

### EXAMPLES

### Example 1: Phylogeny and Protein Alignment

Amino acid sequences of predicted HB (HD-Zip III) family members in Populus trichocarpa genome v3.0 were retrieved from the Phytozome portal (Tuskan et al., 2006) and those from the additional species were retrieved from NCBI. Phylogenetic analysis using the protein sequence information was performed using the Neighbor-Joining method in MEGA (Molecular Evolutionary Genetics Analysis) program using the Neighbor-Joining method (Tamura et al., 2011). Protein sequences were aligned using clustal W program and similarity shading and percent similarity percentage estimation were predicted by GeneDoc program (Nicholas et al., 1997).

### Example 2: RNA Extraction, cDNA Synthesis and qRT-PCR

RNA was extracted from frozen tissue samples using Plant RNA extraction kit (Sigma, St Louis, Mo.) with slight modification as described previously (Payyavula et al., 2014). cDNA was synthesized in a 20 ul reaction using 1.5 μg of total RNA, 1 μl of 100 μM oilgo dT primer and 5.5 μl water, 4 μl buffer, 2 μl of 10 mM dNTP, 0.5 μl of RNAse inhibitor, 1 μl of RevertAid Reverse Transcriptase (Thermofisher). The reaction was incubated at 42° C. for 80 min and heated to 80° C. for 5 min to denature enzymes. Gene expression studies were performed using Quantitative reverse transcriptase PCR (qRT-PCR) in a 20 μl reaction with 3 ng equivalent cDNA, 250 nM primer (Supplemental file) and 10 μl of 2× iTaq Universal SYBR Green (Bio Rad). The expression of housekeeping genes Ubiquitin-conjugating enzyme E2 and 18S ribosomal RNA was used for template normalization. Relative expression of target genes was calculated using delta cT (threshold cycle) or delta-delta cT method (Livak and Schmittgen, 2001).

### Example 3: RNAi Construct Development, GFP-Tagged Protein Localization and Plant Transformation

RNAi construct was developed by amplifying a ˜200 bp nucleotide sequence overlapping the 3′ coding region and UTR regions and ligating in sense and antisense orientation across CHS intron in the vector 156 bp coding region fragment, and ligated in sense and antisense orientation to form hairpin with chalcone synthase intron. The binary constructs was transformed into wild-type P. deltoides ‘WV94’ using Agrobacterium method (Caiping et al., 2004).

Plant Materials:

Transgenic plants and empty-vector transformed control plants that were roughly 15-cm were moved from tissue culture to small tubes with soil. After 2-months, plants were moved to bigger pots (6 liter) and were propagated in a greenhouse maintained at 25° C. at 16 h day length. At the time of harvest (7-month old plants), plant height was measured from shoot tip to stem base, and diameter was measured at base of the stem. In our preliminary study, the bottom 10 cm stem portion was harvested, air-dried and used for carbohydrate composition, cellulose, lignin, S:G ratio, and sugar and ethanol release analysis. Initial studies were performed on 15 to 20 transgenic lines for each construct and additional studies were performed on three to four selected lines. Plants for additional studies were generated from fresh stem cuttings.

For subcellular localization, full length coding region of PdHB3 (Potri.011G098300). was amplified from P. deltoides xylem cDNA library) using Q5 High-Fidelity DNA polymerase (New England Biolabs, Ipswich, Mass.) and cloned in pENTR vector (Invitrogen, Carlsbad, Calif., USA). After sequence confirmation, coding region fragment was recombined into a Gateway binary vector pGWB405 (Tsuyoshi et al., 2009) using LR clonase (Invitrogen). Plasmid from a positive clone was transformed to Agrobacterium tumefaciens strain GV3101. Tobacco infiltration and protein localization was performed as described previously (DePaoli et al., 2011; Sparkes et al., 2006). Agrobacterium harboring binary construct PdHB3 was cultured overnight in LB media. After brief centrifugation, supernatant was removed and pellet was dissolved in 10 mM MgCl₂ and OD at A₆₀₀ was adjusted to 0.5. The culture was infiltrated into 4-week old tobacco leaves. After 48 h, roughly 4 mm² leaf sections were cut and fixed in 3.7% formaldehyde, 50 mM NaH₂PO₄ and 0.2% Triton X-100 for 30 min, rinsed with phosphate-buffered saline (PBS) and stained in DAPI (4,6′-diamidino-2-phenylindole, 1.5 μg ml⁻¹ in PBS) for 30 min. GFP visualization and imaging was performed on Zeiss LSM710 confocal laser scanning microscope (Carl Zeiss Microscopy, Thornwood, N.Y.) equipped with a Plan-Apochromat 63×/1.40 oil immersion objective.

### Example 4: Lignin and Cellulose Quantification

Lignin content and composition from 5 mg of air-dried and ground stem material was estimated by pyrolysis molecular beam mass spectrometry (MBMS) at National Renewable Energy Laboratory as described previously (Mielenz et al., 2009). Cellulose content was measured using anthrone method (Updegraff, 1969). Briefly, 25 mg of dried stem sample was incubated at 98° C. for 30 min with 0.5 ml of acetic acid-nitric acid reagent (72.7% acetic acid 9.1% nitric acid). After brief centrifugation, supernatant was discarded and pellet was washed with water. The pellet was then digested for 60 min with 67% (v/v) sulfuric acid. Glucose released from cellulose digestion was then estimated using cold anthrone solution as described previously.

### Example 5: Structural Carbohydrate Composition and Ethanol Conversion Analysis

Structural carbohydrate analysis was performed on air-dried stem material as described previously. Soluble sugars and starch from 25 mg sample were eliminated by extracting the sample with 80% ethanol followed by digestion with α-amylase (from Aspergillus oryzae, Sigma) and amyloglucosidase (from Aspergillus niger, Sigma). The dried starch free sample was digested for 1 hr with H₂SO₄ (72% v/v). After dilution with water, the samples were autoclaved for 1 hr, cooled and neutralized with CaCO₃. The sugar monomers were then analyzed on high performance liquid chromatography (HPLC, LaChrom Elite® system, Hitachi High Technologies America, Inc.) as described previously (Fu et al., 2011; Yee et al., 2012). Glucose release and ethanol conversion from stem biomass were estimated using Separate hydrolysis and fermentation (SHF) as described previously (Fu et al., 2011; Yee et al., 2012).

### Example 6: Results

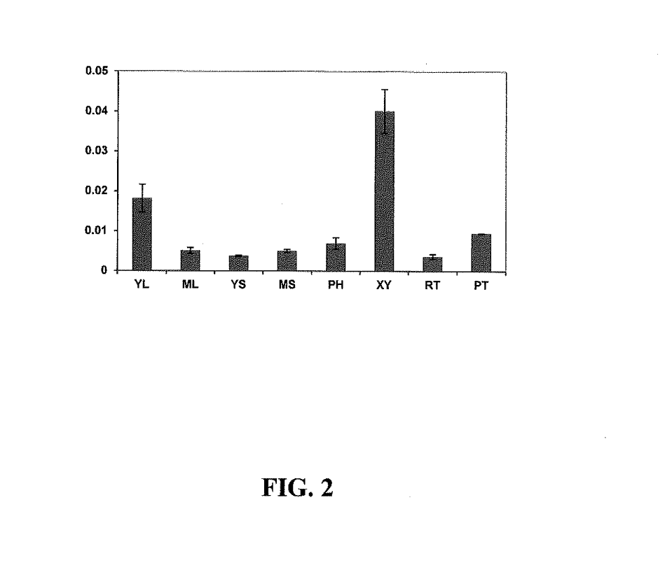

PdHB31 is expressed at low level with a relative enhanced expression in xylem tissue (FIG. 2).

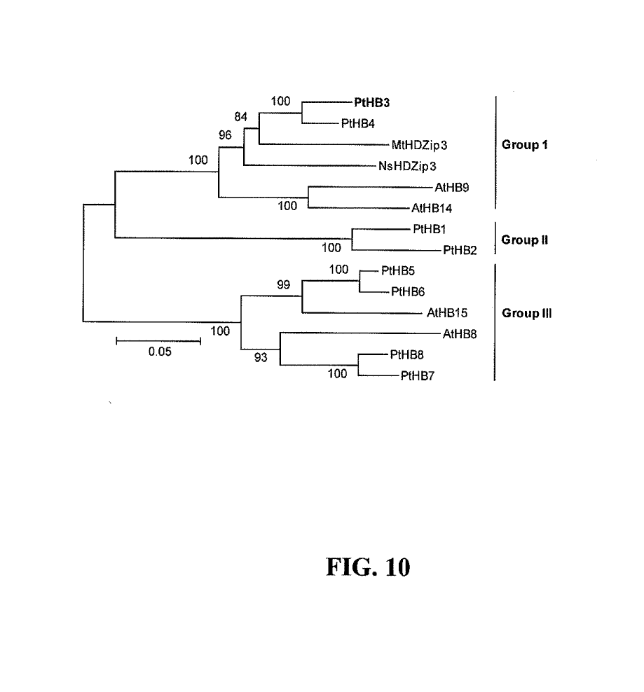

PdHB3 is predicted to code for a transcription factor protein belonging to the HDZipIII transcription factor family. PdHB3 predicted protein sequence is closely related to that of Arabdopsis homologs, AtHB14 and AtHB9. Phylogenetic analysis of HB3 proteins was developed using neighbor joining method. In the schematic shown in FIG. 10, the closely related HB3 paralogs are shown in a box and the PdHB31, highlighted in bold was the target in this study. Phylogenetic analysis of HDZipIII transcription factors in Populus and Arabidopsis developed using neighbor joining method. AtATHB8, At4g32880 (Arabidopsis thaliana); AtATHB9, AT1G30490; AtATHB14, AT2G34710; AtATHB-15, At1G52150; MtHDZip3, AES64771 (Medicago truncatula); NsHDZip3, AAS66760 (Nicotiana sylvestris); PtHB1, Potri.009G014500 (Populus trichocarpa); PtHB2, Potri.004G211300; PtHB3, Potri.011G098300; PtHB4, Potri.001G372300; PtHB5, Potri.001G188800; PtHB6, Potri.003G050100; PtHB7, Potri.018G045100; PtHB8, Potri.006G237500. PtHB3, highlighted in bold was the target in this study.

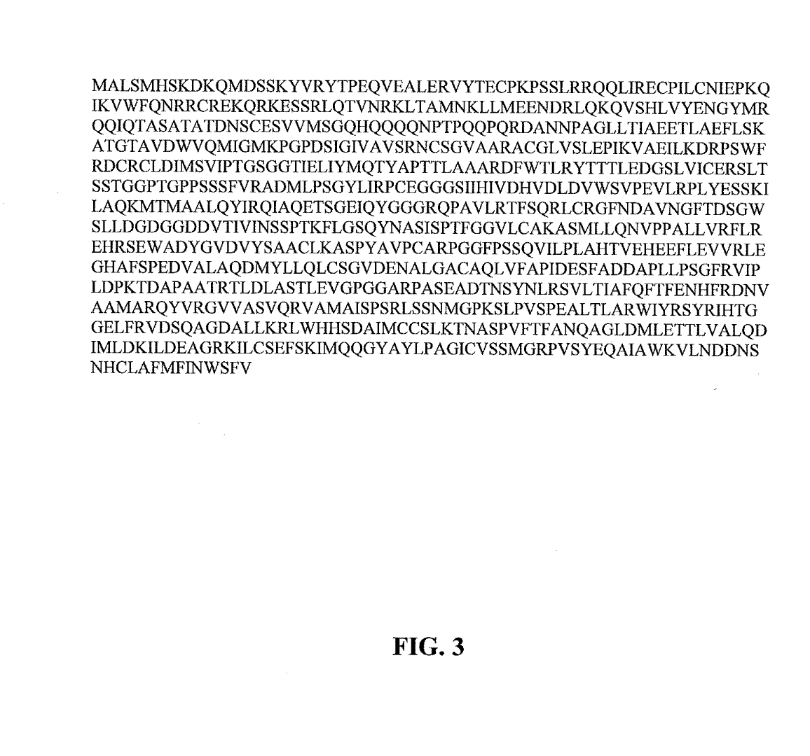

The full-length protein sequence of PdHB3 (Potri.011G098300) consists of 302 AA (SEQ. ID. NO: 2) and is shown in FIG. 3.

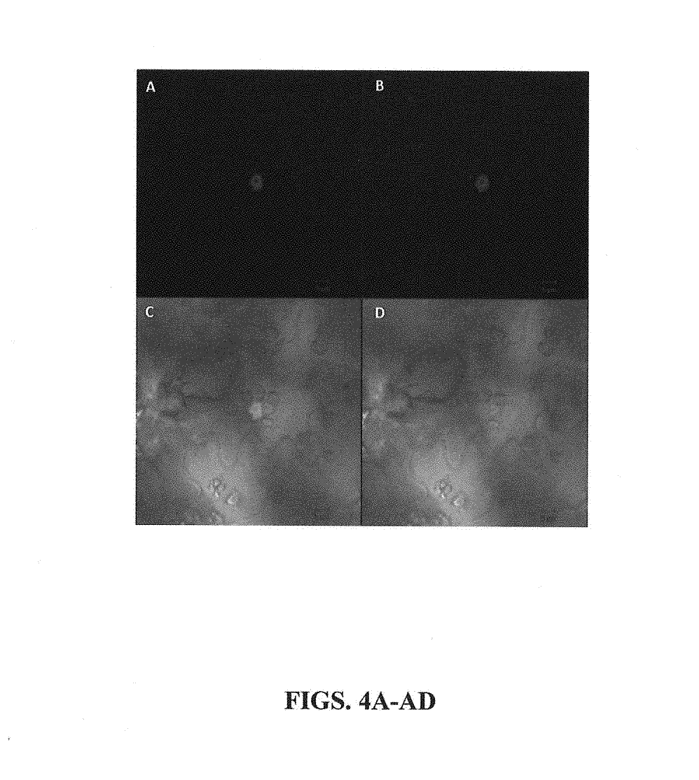

FIG. 4 shows the subcellular localization of PdHB3 protein. Results from tobacco leaf infiltration using constructs developed to probe subcellular localization of PdHB3 protein. FIG. 4A shows fluorescence from DAPI-stained nucleus as a positive control. FIG. 4B shows the fluorescence from GFP-tagged PdHB3. FIG. 4C is an overlay of FIGS. 4A and 4B and confirms nuclear localization of PdHB3 transcription factor. FIG. 4D is a white light image of tobacco mesophyll cells.

To generate transgenic RNAi plants, stable down-regulation of PdHB3 gene target was achieved using RNAi gene silencing technology in combination with agrobacterium mediated transformation of plant cells. Other alternate technologies for achieving RNA silencing include antisense and artificial miRNA (amiRNA) technologies.

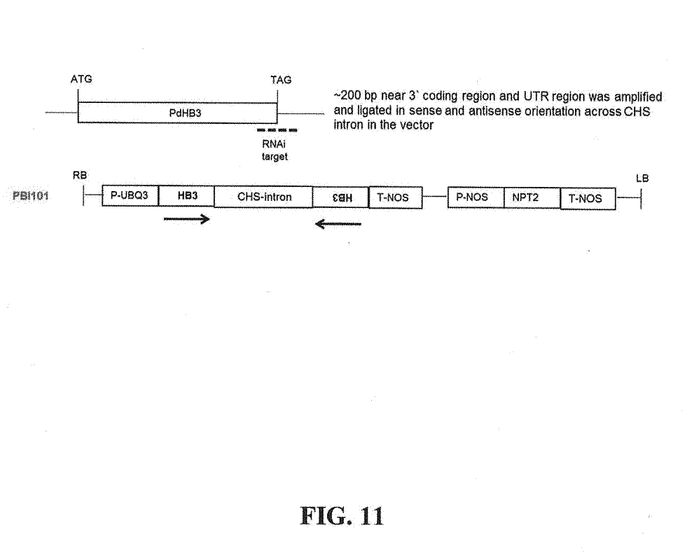

The construct design, shown in FIG. 11, was used to develop the HB3 gene-specific RNAi construct.

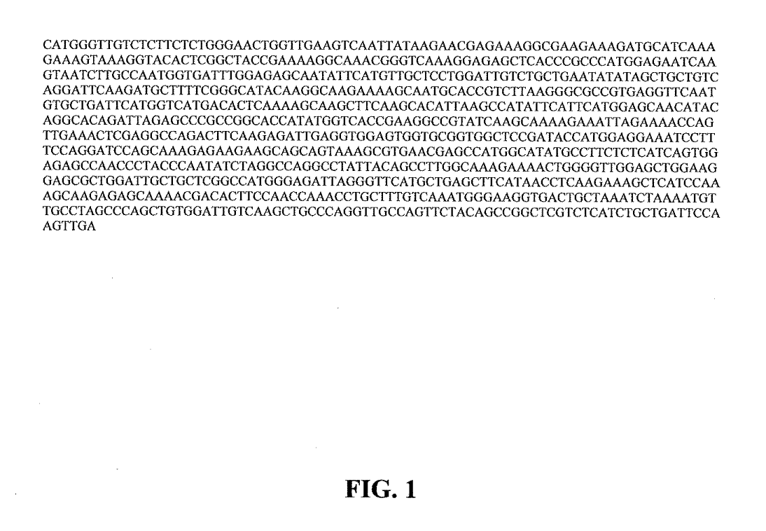

See FIG. 1 for Potri.011G098300 (SEQ. ID NO: 1), Coding sequence (translated region) from reference genome, P. trichocarpa nisqually1).

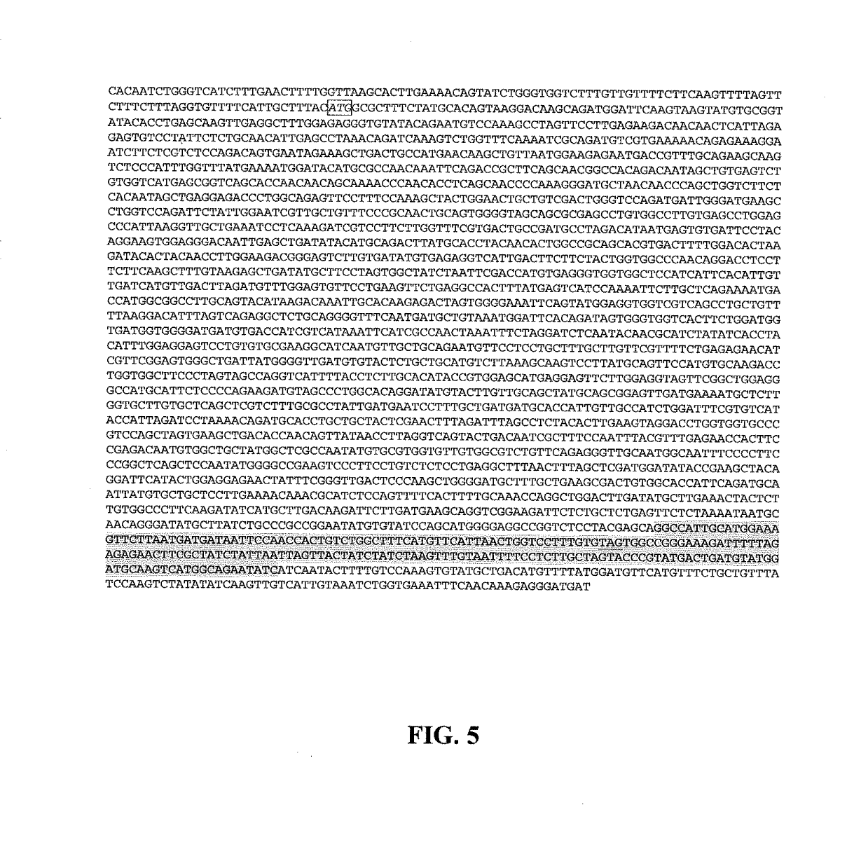

Gene-specific RNAi construct targeted to Populus deltoides PdHB3 gene (Gene model: Potri.011 G098300) expressed under the Polyubiquitin 3 (AtUBQ3, Accession L05363) promoter. Full gene nucleotide sequence (SEQ. ID. NO: 3) of Potri.011G098300 is provided below (RNAi target region spanning the 3′ translated and untranslated regions is highlighted in grey (“PdHB3” insert in the construct design). Start codon is highlighted in a box and in italics and stop codon is underlined). See FIG. 5.

Primers used for RNAi construct development:forward primer: CACCCCCGGGCCATGCATGATGAAAGTTCT (SEQ. ID. NO: 4) and Reverse primer: TCTAGAGATATTCTGCCATGACTTGCATC (SEQ. ID. NO: 5). P. deltoides libraries were used for amplification of target.

The sequence-specific down-regulation of the one target gene (HB3) was verified by studying the relative expression (arbitrary units) of another paralogous gene closely related to PdHB3 (PdHB4) in control and HB3 transgenic lines. Our assays confirmed that while downregulation in expression level of the target gene was confirmed (FIG. 6), the expression of other PdHB3 genes was not significantly altered in two independent PdHB3 transgenic RNAi lines, HB3-1 and HB3-2. This provides evidence that the observed transgenic phenotype is due to the downregulation of expression of PdHB3.

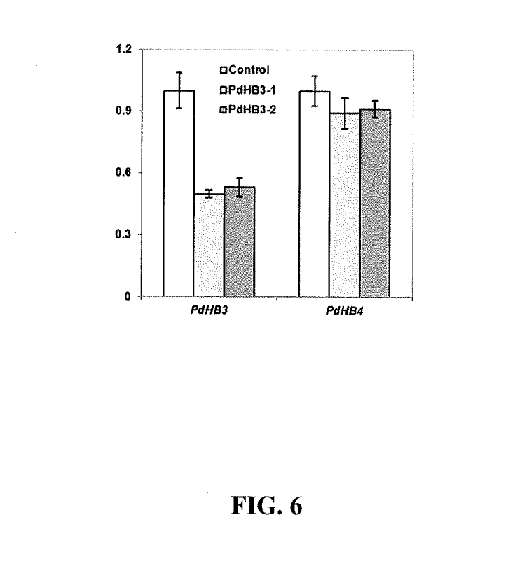

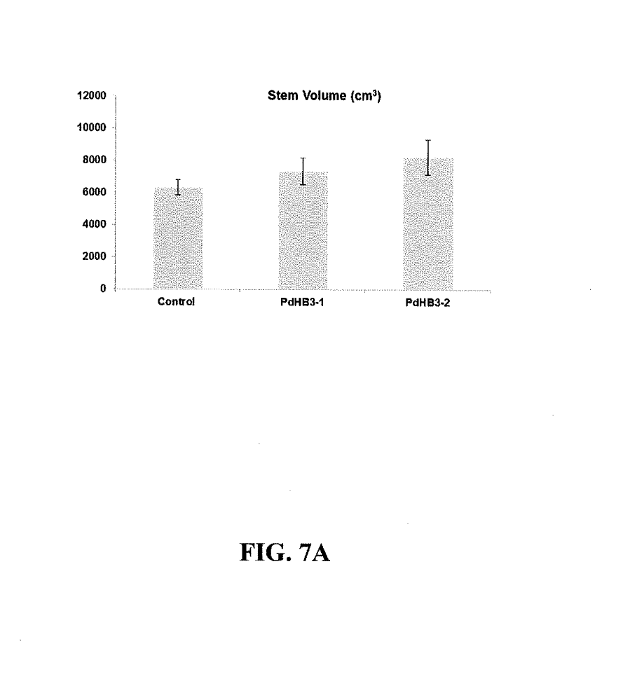

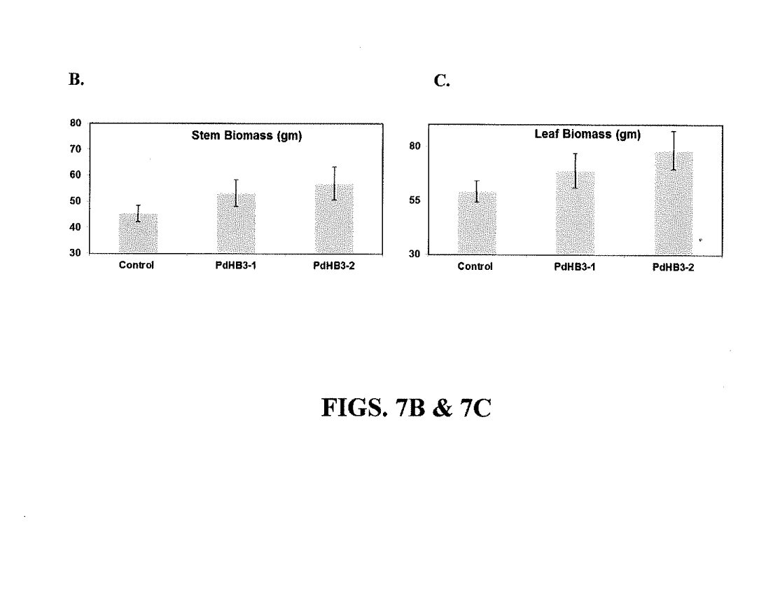

Greenhouse-grown PoptrHB3 transgenic RNAi plants showed increased plant growth and stem biomass formation (FIGS. 7A, 7B, and 7C).

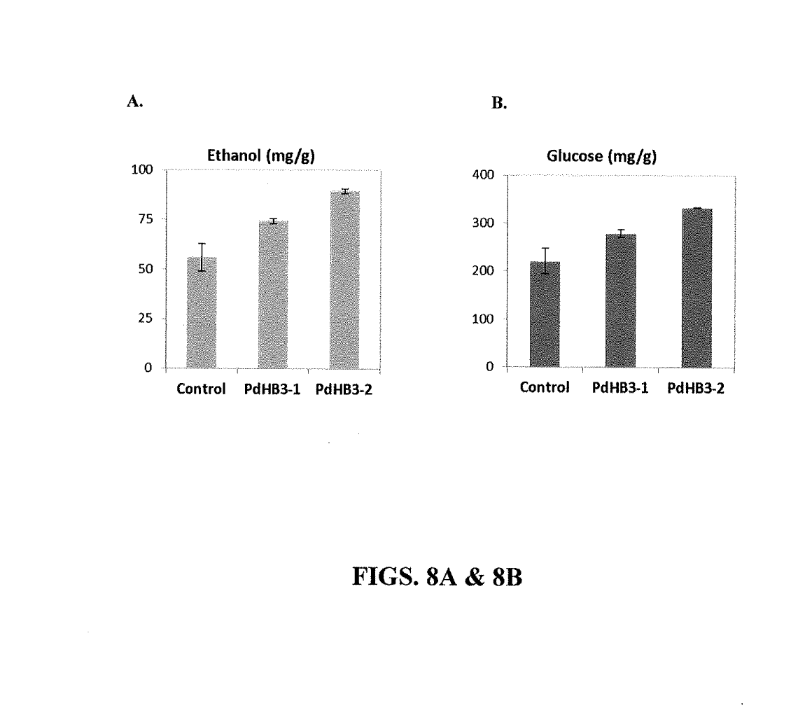

Greenhouse-grown PoptrHB3 transgenic RNAi plants showed lower recalcitrance and better sugar release properties. The PdHB3 transgenic lines showed a significant increase in glucose release (FIG. 8B) and ethanol conversion (FIG. 8A). Line PdHB3-1 showed 26% and 32% increase in glucose release and ethanol conversion, respectively; whereas line PdHB3-2 showed 50% and 60% increase in glucose release and ethanol conversion, respectively, as compared to empty vector transformed control plants.

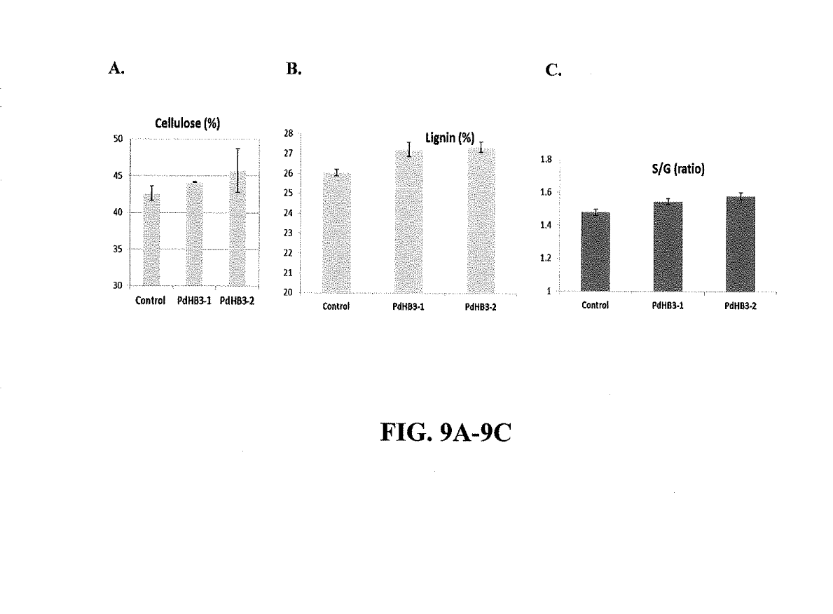

PoptrHB3 RNAi plants showed altered cell wall composition with higher lignin (FIG. 9A) and cellulose content (FIG. 9B).

### REFERENCES

Endler A, Persson S. Cellulose synthases and synthesis in Arabidopsis. Mol. Plant. 2011; 4:199-211.
Mizrachi E, Mansfield S D, Myburg A A. Cellulose factories: advancing bioenergy production from forest trees. New Phytol. 2012; 194:54-62.
Kalluri U C, Yin H, Yang X, Davison B H. Systems and synthetic biology approaches to alter plant cell walls and reduce biomass recalcitrance. Plant Biotechnol J. 2014 December; 12(9):1207-16. doi: 10.1111/pbi.12283. Epub 2014 Nov. 3.
Payyavula R S, Tschaplinski T J, Jawdy S S, Sykes R W, Tuskan G A, Kalluri U C. Metabolic profiling reveals altered sugar and secondary metabolism in response to UGPase overexpression in Populus. BMC Plant Biol. 2014 Oct. 7; 14:265. doi: 10.1186/s12870-014-0265-8.
Hu R, Chi X, Chai G, Kong Y, He G, et al. (2012) Genome-Wide Identification, Evolutionary Expansion, and Expression Profile of Homeodomain-Leucine Zipper Gene Family in Poplar (Populus trichocarpa). PLoS ONE 7(2): e31149

What is claimed is:

1. A genetically modified plant characterized by a reduced expression of the PdHB3 gene as compared to a control plant.

2. The genetically modified plant of claim 1, wherein said genetically modified plant belongs to a genus selected from the group consisting of Populus, Arabidopsis, Medicago, and Nicotiana.

3. The genetically modified plant of claim 2, wherein the plant is selected from the group consisting of of Populus balsamifera, Populus deltoides, Populus trichocarpa, Populus tremuloides, Populus tremula, Populus alba.

4. The genetically modified plant of claim 1, wherein the reduced expression of the PdHB3 gene is achieved by introducing a nucleic acid inhibitor.

5. The genetically modified plant of claim 4, wherein said nucleic acid inhibitor is selected from the group consisting of an antisense RNA, a small interfering RNA, an RNAi microRNA, an artificial microRNA, and a ribozyme.

6. A method for biofuel production, comprising using the plant of claim 1 in a biofuel fermentation process.

7. An expression vector comprising a nucleotide sequence that is transcribed into a nucleic acid inhibitor of expression of the PdHB3 gene, operably linked to a regulatory region that is functional in a plant, wherein the nucleic acid is selected from the group consisting of an antisense RNA, a small interfering RNA, an RNAi microRNA, an artificial microRNA, and a ribozyme.

8. The expression vector of claim 7, wherein the regulatory region comprises an inducible promoter or a tissue-specific promoter.

9. The expression vector of claim 8, wherein the tissue-specific promoter is a xylem-specific promoter.

10. A method for increasing glucose and/or xylose release in a plant or plant cell, comprising introducing the expression vector of claim 7 in said plant or plant cell, and expressing the nucleic acid in plant or plant cell.

11. A plant or plant cell comprising the expression vector of claim 7.

12. A method of selecting a plant for improved biomass characteristics comprising:
a. obtaining nucleic acids from a candidate plant;
b. detecting the presence of an allelic variant of the PdHB3 gene in said nucleic acids; and
c. selecting said plant based on the presence of an allelic variant of the PdHB3 in the nucleic acids obtained from the plant.

13. A method according to detect the presence of an allelic variant of PdHB3 gene in a plant, comprising identifying a plant having increased biomass and ethanol production, and determining the sequence of the gene at the PdHB3 locus in said plant.
